(19) United States
(12) Patent Application Publication
(10) Pub. No.: US 2010/0303739 A1
(43) Pub. Date: Dec. 2, 2010

(54) Highly Concentrated Fatty Alcohol Sulfate Preparation

(75) Inventors: Roland Spoerer, Atizadan de Zaragoza (MX); Srarat Jariyachat, Chonburi 20000 (TH); Tedy Waworuntu, Bogor 16968 (ID); Achmad Jazuli, Jawa Barat (ID); Achmad Taufik, Mampang Depok 16433 (ID)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/678,041

(22) PCT Filed: Sep. 6, 2008
(86) PCT No.: PCT/EP08/07295

§ 371 Date: Mar. 12, 2010

(30) Foreign Application Priority Data
Sep. 14, 2007 (EP) 07018144.1

(51) Int. Cl. A61K 8/46; A61Q 11/00; A61Q 5/02; C11D 1/14

(52) U.S. Cl. 424/54; 424/56; 510/127; 510/536

(57) ABSTRACT

Concentrated fatty alcohol sulfate preparations which comprise at least 35% by weight of fatty alcohol sulfate (FAS) are described. The compositions are aqueous, but, besides the FAS, also comprise polyols, fatty acid alkylamides and/or oil-soluble betaines. They are suitable for the production of detergents and cleaners, but also for the production of cosmetic compositions, in particular dental care compositions.

The present application relates to concentrated surfactant preparations based on alkyl sulfate surfactants, to the use thereof and to the production thereof.

Fatty alcohol sulfates are known anionic surfactants which are used in a multitude of detergents and cleaners, but also in cosmetic compositions, e.g. in shampoos, skin cleansing compositions, skin soaps, but also in toothpastes.

The alkyl sulfates are produced industrially, generally by sulfation of suitable fatty alcohols with gaseous SO₃. The alkylsulfuric acid which forms as crude product in the process is then neutralized in the presence of suitable alkalis, preferably aqueous sodium hydroxide, to give the alkyl sulfates. At the end of such processes, surfactant-containing aqueous preparations with a maximum of 30% by weight of anionic surfactants are obtained.
To transport such preparations, the water is often removed in order to obtain dry products, which are easier to transport. However, the removal of the water constitutes a process step that consumes a lot of energy and is therefore expensive. Transportation of the directly produced aqueous compositions themselves is not advantageous; in so doing only comparatively small amounts of active substance (maximum 30% by weight) but large amounts of water are transported. Moreover, the processing of dried fatty alcohol sulfates is time-consuming. The use of liquid fatty alcohol sulfates generally results in a batch time reduction of more than 20% on the part of the manufacturer of shampoos, skin cleansing compositions, skin soaps but also in toothpastes.

There is therefore a need to provide pumpable, liquid or slightly pasty, highly concentrated fatty alcohol sulfate preparations in order to avoid the problems described above.

It has been found that relatively high-concentration, pumpable, i.e. low viscosity, fatty alcohol preparations can be obtained by adding certain additives.

The application therefore firstly provides compositions comprising at least

(a) 35% by weight, based on the total weight of the composition, of one or more fatty alcohol sulfates of the general formula (I)

R¹O —SO₃X (I)

in which R¹ is a linear or branched, aliphatic alkyl and/or alkenyl radical having 6 to 22, preferably 12 to 18, carbon atoms and X is an alkyl metal and/or alkaline earth metal, ammonium, alkylammonium, alkanolammonium or glucammonium,
(b) a polyol and/or a betaine and/or a fatty acid alkylamide and
(c) water.

Alkyl sulfates and/or alkenyl sulfates, which are often also referred to as “fatty alcohol sulfates”, are to be understood as meaning the sulfation products of primary alcohols which conform to the formula (I)

R¹O—SO₃X (I)

in which R¹ is a linear or branched, aliphatic alkyl and/or alkenyl radical having 6 to 22, preferably 12 to 18, carbon atoms and X is an alkali metal and/or alkaline earth metal, ammonium, alkylammonium, alkanolammonium or glucammonium. Typical examples of alkyl sulfates which can be used within the context of the invention are the sulfation products of caproic alcohol, caprylic alcohol, capric alcohol, 2-ethylhexyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol and erucyl alcohol, and technical-grade mixtures thereof which are obtained from high-pressure hydrogenation of technical-grade methyl ester fractions or aldehydes from the Roelen oxo synthesis. The sulfation products can preferably be used in the form of their alkali metal salts and in particular their sodium salts.

Particular preference is given to alkyl sulfates based on C₁₆/₁₈-tallow fatty alcohols and/or vegetable fatty alcohols of comparable carbon chain distribution in the form of their sodium salts. Alkyl sulfates based on dodecyl alcohol may be of particular importance since these sulfates are used in dental care and dental cleansing compositions. However, preference is furthermore also given to fatty alcohol cuts from fatty alcohols having 12 to 14 carbon atoms, in which case, it may be particularly preferred here to use those cuts which only comprise very small fractions of C16 fatty alcohols, e.g. less than 10% by weight, preferably less than 5% by weight and in particular less than 3% by weight, based on the amount of fatty alcohols. Particular preference is furthermore given to the saturated fatty alcohols and here in turn to the abovedescribed cuts.

The compositions according to the invention are distinguished by the fact that they are highly concentrated, i.e. in the context of this application that they comprise at least 35% by weight of fatty alcohol sulfates, based on the total composition. The compositions according to the claimed technical teaching can, however, preferably comprise fatty alcohol sulfates in amounts, based on the total weight of the composition, of more than 35% by weight, preferably of from 36% by weight to 70% by weight, in particular 40 to 60% by weight and particularly preferably from 40 to 50% by weight.

An essential feature of the invention is the presence of polyols, or the betaines and/or the fatty acid alkylamides, although from this group of three, the polyols are the preferred choice.

Polyols which are suitable here have preferably 2 to 15 carbon atoms and at least two hydroxyl groups. The polyols can also contain further functional groups, in particular amino groups, and/or be modified with nitrogen. Typical examples are glycerol; alkylene glycols, such as, for example, ethylene glycol, diethylene glycol, propylene glycol, butylene glycol, hexylene glycol, and polyethylene glycols with an average molecular weight of from 100 to 1000 daltons; technical-grade oligoglyceryl mixtures with a degree of self-condensation of from 1.5 to 10, such as, for example, technical-grade diglycerol mixtures with a diglycerol content of from 40 to 50% by weight; methylol compounds, such as, in particular, trimethylolethane, trimethylolpropane, trimethylolbutane, pentaerythritol and dipentaerythritol; lower alkyl glucosides, in particular those with 1 to 8 carbon atoms in the alkyl radical, such as, for example, methyl glucoside and butyl glucoside; sugar alcohols with 5 to 12 carbon atoms, such as, for example, sorbitol or mannitol; sugars with 5 to 12 carbon atoms, such as, for example, glucose or sucrose; amino sugars, such as, for example, glucamine; dialcohol amines, such as diethanolamine or 2-amino-1,3-propanediol. Preferred polyols are selected from the group glycerol, ethylene glycol, propylene glycol, or sorbitol, with glycerol and sorbitol being particularly preferred.

Betaines are known surfactants which are produced predominantly by carboxyalkylation, preferably carboxymethylation, of aminic compounds. Preferably, the starting materials are condensed with halocarboxylic acids or salts thereof, in particular with sodium chloroacetate, with one mole of salt being formed per mole of betaine. Furthermore, the addition of unsaturated carboxylic acids, such as, for example, acrylic acid, is also possible. Particular preference is given to betaines which are obtained by reacting oils, i.e.

triglycerides. An example which may be given is N,N-dimethyl-N-(cocoamidopropyl)ammonium acetobetaine (Dehyton PK 45-Cognis).

Preference is given to oil-soluble betaines.

Fatty acid alkylamides are likewise compounds that are known per se. They are also understood to include fatty acid alkanolamides in the narrow sense, with the fatty acid di- or monoethanolamides being particularly preferred here. In this connection, preference is furthermore given to compounds of this type comprising saturated or unsaturated, branched or unbranched fatty acids with carbon chain lengths of from 7 to 21. These are sold, for example, by the applicant under the name

“Comperlan”. Particular preference is given to fatty acid alkylamides based on coconut fatty acid mono-ethanolamides. It seems that oil-soluble fatty acid alkylamides are particularly preferred.

The polyols, but also the betaines and/or the fatty acid alkylamides, are present in the compositions in amounts, based on the total weight of the compositions, of from 10 to 45% by weight, preferably 15 to 40% by weight and in particular 25 to 35% by weight. It may be preferred to use mixtures of polyols, betaines and/or fatty acid alkylamides.

The compositions according to the invention obligatorily comprise the fatty alcohol sulfates, polyols or betaines or the fatty acid alkylamides and water, but can moreover also comprise other ingredients, e.g. inorganic salts or nonionic surfactants. However, particular preference is given to compositions which consist only of the three specified components.

The aqueous compositions preferably have a pH greater than 8, the pH preferably being in the range from 9 to 12, in particular preferably in the range from 10 to 12. The compositions can be in the form of a liquid or in the form of a paste, preferably in the form of a low viscosity, pumpable paste. Typical viscosities, measured in accordance with Brookfield at 25° C. (spindle 3 or 4, 10 rpm) are 500 to 10 000 mPas and preferably 1000 to 8000 mPas.

Based on the weight of the total composition, a preferred composition within the context of the present invention comprises (a) 36 to 45% by weight of alkyl sulfates of the formula (I), (b) 28 to 45% by weight of polyols, where glycerol and/or sorbitol are preferred, or betaines and/or fatty acid alkylamides and (c) 10 to 34% by weight of water.

The concentrated fatty alcohol preparations are preferably produced by neutralizing the sulfuric-acidic fatty alcohol ester in the presence of a water/polyol or betaine mixture. A further subject matter therefore relates to a method for producing a preparation, comprising at least 35% by weight of alkyl sulfates of the formula (I) according to the above description, where an unneutralized alkyl sulfate of the general formula R¹—OSO₃H is continuously added together with an amount of alkalis to a mixture of (A) water and (B) a polyol or betaine—where the volume ratio of (A) to (B) is adjusted to values of from 3:1 to 1:3 —where the amount of alkalis is suitable to completely neutralize the alkyl sulfate, and the mixing of the mixture of (A) and (B) with the unneutralized alkyl sulfate and the alkalis is continued until at least a concentration of neutralized alkyl sulfates of the formula (I) of 35% by weight has been reached.

Sulfation of the fatty alcohols takes place in a known manner with SO₃. The crude sulfation product is then metered into the mixture of (A) and (B) and at the same time an aqueous alkali metal hydroxide solution, preferably aqueous sodium hydroxide, is preferably metered in separately so that simultaneous mixing and neutralization takes place. This procedure is carried out until the desired concentration range of the fatty alcohol sulfate preparation is achieved. Then, if desired, further additives or ingredients may also be metered in. The process product can then, for example, however also be bleached and/or provided with fragrances. It may be advantageous to vary the quantitative ratio of (A) and (B) among one another, where a volume ratio in the range from 3:1 to 1:1 and in particular from 2:1 to 1.5:1 may be particularly preferred.

A further subject matter relates to the use of a composition according to the above description for producing detergents or cleaners, or cosmetic compositions, for example hair shampoos or toothpastes or similar care products. Detergents, cleaners and/or cosmetic compositions which comprise the concentrated alkyl sulfates within the context of the present teaching are likewise subject matter for which protection is sought.

### EXAMPLES

A fatty alcohol mixture with the qualitative composition C12: 65-71% by weight; C14: 22-28% by weight; C16: 3-6% by weight was sulfated in an SO₃ plant. The sulfated crude product was then continuously metered into a mixture of polyols with water in the weight ratio of 3:1 together with a 30% strength aqueous NaOH solution in a stainless steel neutralization container. After neutralization had taken place, the end product was discharged. It had a content of sulfated fatty alcohols of 42% by weight. The pH was 11. The color (APHA, 20% strength by weight solution) was measured as 50.

In this way, products (fatty alcohol sulfate according to the abovedescribed qualitative composition of the fatty alcohol mixture) with the following compositions (all data in % by weight) were prepared:

| | Fatty alcohol | Glyc- | Sor- | Beta- | Fatty acid | |
|---|---|---|---|---|---|---|
| No. | sulfate | erol | bitol | ine¹⁾ | alkylamide²⁾ | Water |
| 1 | 30 | 40 | | | | 60 |
| 2 | 40 | 26 | 19 | | | 85 |
| 3 | 35 | 26 | | 10 | | 29 |
| 4 | 35 | 26 | | | 10 | 29 |

¹⁾Dehyton PK 45
²⁾Comperlan CMEA

What is claimed is:

1. A composition comprising:
(a) at least 35% by weight, based on the total weight of the composition, of one or more fatty alcohol sulfates of formula (I)
R¹O—SO₃X (I)
in which R¹ is a linear or branched, aliphatic alkyl or alkenyl group having 6 to 22 carbon atoms, and X is selected from the group consisting of alkali metals, alkaline earth metals, ammonium, alkylammonium, alkanolammonium, glucammonium, and combinations thereof,
(b) at least one additive selected from the group consisting of polyols, fatty acid alkylamides, betaines and combinations thereof; and
(c) water.

2. The composition of claim 1, comprising from 36% to 70% by weight of said fatty alcohol sulfates, based on the total weight of the composition.

3. The composition of claim 1 wherein said polyol is selected from the group consisting of glycerol, ethylene glycol, propylene glycol, sorbitol and mixtures thereof.

4. The composition of claim 1 comprising from 10% to 45% by weight of component (b), based on the total weight of the composition.

5. The composition of claim 1 comprising from 5% to 40% by weight of water, based on the total weight of the composition.

6. The composition of claim 1, comprising:
(a) 36 to 45% by weight of one or more alkyl sulfates of formula (I),
(b) 28 to 45% by weight of at least one additive selected from the group consisting of polyols, betaines, fatty acid alkylamides, and combinations thereof; and
(c) 10 to 34% by weight of water, based on the weight of the total composition.

7. The composition of claim 1 in the form of a liquid or low-viscosity pumpable paste.

8. (canceled)

9. A method of producing detergents, cleaners, or cosmetic compositions, comprising adding the composition of claim 1 to an appropriate detergent, cleaner, or cosmetic base.

10. A detergent, cleaner or cosmetic composition comprising the fatty alcohol sulfate preparation of claim 1.

11. A method for producing a highly concentrated fatty alcohol sulfate preparation, comprising the steps of:
(a) providing a mixture of
(A) water, and
(B) at least one polyol or oil-soluble betaine, wherein the volume ratio of (A) to (B) is from 3:1 to 1:3; and
(b) simultaneously adding, with continuous mixing
(i) unneutralized alkyl sulfate of formula R¹—OSO₃H, and
(ii) sufficient alkali to completely neutralize the alkyl sulfate (i), until a concentration of neutralized alkyl sulfates of at least 35% by weight has been achieved.

12. The method of claim 9, wherein said composition is added to a cosmetic base to produce a hair shampoo.

13. The method of claim 9, wherein said composition is added to a dentifrice base to produce a toothpaste.

14. The composition of claim 1, wherein R¹ is a linear or branched, aliphatic alkyl or alkenyl group having 12 to 18 carbon atoms.

15. The composition of claim 2, comprising from 40% to 50% by weight of said fatty alcohol sulfates, based on the total weight of the composition.

16. The composition of claim 3, wherein said polyol is selected from the group consisting of glycerol, sorbitol and combinations thereof.

17. The composition of claim 4, comprising from 25% to 35% by weight of component (b), based on the total weight of the composition.

18. A composition consisting of:
(a) at least 35% by weight, based on the total weight of the composition, of one or more fatty alcohol sulfates of formula (I)
R¹O—SO₃X (I)
in which R¹ is a linear or branched, aliphatic alkyl or alkenyl group having 6 to 22 carbon atoms, and X is selected from the group consisting of alkali metals, alkaline earth metals, ammonium, alkylammonium, alkanolammonium, glucammonium, and combinations thereof,
(b) at least one additive selected from the group consisting of polyols, fatty acid alkylamides, betaines and combinations thereof; and
(c) water.
